(19) United States
(12) Patent Application Publication
(10) Pub. No.: US 2009/0112199 A1
(43) Pub. Date: Apr. 30, 2009

(54) EP SIGNAL MAPPING-BASED OPTICAL ABLATION FOR PATIENT MONITORING AND MEDICAL APPLICATIONS

(75) Inventors: Hongxuan Zhang, Schaumburg, IL (US); Detlef W. Koertge, Carpentersville, IL (US); Harold James Wade, Rockford, IL (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Medical Solutions USA,Inc., Malvern, PA (US)

(21) Appl. No.: 12/202,478

(22) Filed: Sep. 2, 2008

Related U.S. Application Data
(60) Provisional application No. 60/982,511, filed on Oct. 25, 2007.

(51) Int. Cl. A61B 18/24

(52) U.S. Cl. 606/15

(57) ABSTRACT

A system and method are disclosed for utilizing a single integrated EP/ablation catheter to treat cardiac arrhythmias. The disclosed catheter combines the EP signal monitoring of a traditional EP diagnostic catheter, and optical energy for the ablation therapy, which is expected to provide a more efficient, accurate and reliable method of cardiac ablation than current RF techniques since it is based on real-time EP signal mapping, with precise pathological tissue localization, cardiac arrhythmia severity characterization and delivers predictable energy doses with continuous safety monitoring of the intracardiac signals.

### CROSS-REFERENCE TO RELATED APPLICATIONS

This is a U.S. non-provisional application of pending U.S. provisional patent application Ser. No. 60/982,511 to Hongxuan Zhang, et al., filed Oct. 25, 2007, titled “EP Signal Mapping Based Optical Ablation in the Patient Monitoring and Medical Application,” the entirety of which application is incorporated by reference herein.

### FIELD OF THE DISCLOSURE

The disclosure relates to systems and methods for cardiac ablation, and more particularly to a system and method for integrating EP signal mapping and cardiac ablation functionality into a single catheter.

### BACKGROUND OF THE DISCLOSURE

Cardiac ablation is an important invasive treatment modality used to treat many types of heart arrhythmia, which includes atrial fibrillation (AF), atrial flutter, AV re-entrant tachycardia, accessory pathway arrhythmias (such as WPW) and ventricular tachycardia. Typically, a radio frequency (RF) energy ablation catheter is used in the electrophysiology (EP) laboratory to treat the patient's arrhythmia by destroying pathological conduction tissue of the heart and thus returning the patient to a healthy rhythm and/or preventing a patient from going into an unhealthy heart rhythm. The RF energy is directed through the catheter to small areas of the heart muscle that are causing and/or conducting the abnormal heart rhythm. This energy “disconnects” the underlying aberrant pathway of the abnormal cardiac rhythm. In some cases, there is a need to disconnect the electrical pathway between the upper chambers (atria) and the lower chambers (ventricles) of the heart to insert a pacemaker. Ablation energy is used for this procedure as well.

During the ablation procedure, in order to get a precise arrhythmia diagnosis and accurate localization, an EP catheter with multiple leads is employed to capture intra-cardiac signals. Currently, there is no single catheter available that integrates both EP signal acquisition and ablation functions with satisfactory signals. This is because the RF ablation energy delivery can generate large amounts of electrical noise, which saturates the EP signals and makes accurate and precise data capture difficult or impossible.

Recent research demonstrates that optical energy can be utilized for pulmonary vein isolation in treatment of atrial fibrillation, for example using CardioFocus Inc.'s Endoscopic Ablation System. However, there are still several shortcomings and areas for improvement with the current methods and strategies of EP monitoring and energy ablation for clinical applications.

One downside of current systems is that current clinical applications of multi-channel EP signal monitoring and intra-cardiac energy ablation delivery require the use of two different catheters: an EP signal acquisition (diagnostic) catheter and an ablation catheter. The multi-catheter insertion procedure may be time consuming, which unavoidably increases the potential risk to heart tissue and circulation system.

Additionally, current clinical treatments for cardiac arrhythmia typically utilize an EP signal acquisition catheter and an ablation catheter at the same time. (With such procedures, the EP signal acquisition catheter is used as an approximate timing and site reference, not to provide a precise tissue site location.) In such cases heart contractions and myocardial tissue movement may shift the relative position between the EP catheter and the ablation catheter, which may lead to tissue localization deviation resulting in unnecessary false ablation.

Furthermore, current clinical ablation treatments for cardiac arrhythmias typically utilize RF energy for termination of rhythmic disorders and irregularities and electrical pathway isolation. The use of RF energy, however, has certain shortcomings: (1) it may generate unnecessary noise that can interfere with other sensors and signal monitoring, such as EP signal and intra-cardiac ultrasound signals; (2) RF energy delivery is an electrical shock and energy discharge procedure which may affect large amounts of cardiac tissue resulting in imprecise and inefficient ablation; (3) RF energy may be delivered anisotropically (ie., energy is conducted and distributed unevenly to the cardiac tissue) which may cause unnecessary tissue burning, unwanted secondary myocardial injury, and even unsuccessful ablation to the pathological tissue; and (4) often affected patients have implanted cardiac rhythm management devices, which may be harmed and/or reprogrammed by applied RF energy.

In addition, EP signal mapping and pathology characterization using an EP catheter are not well utilized to provide information for ablation decision-making such as accurate localization, arrhythmia type/severity, ablation point priorities, delivering energy estimation, ablation launch time, and the like.

Finally, current ablation procedures focus on single point energy delivery. Recent studies and medical research indicates that simultaneous multi-site ablation and sequential multi-site ablation based on EP mapping and characterization may provide more efficient arrhythmia ablation (i.e., it may be less time consuming with higher success rates), even for persistent cardiac arrhythmias.

Thus, there is a need for an improved ablation system in which EP signal acquisition and ablation functions are integrated into a single catheter. In addition, there is a need for an improved mapping and ablation system that can accommodate simultaneous and/or sequential multi-site ablation based on EP tissue mapping and characterization.

### SUMMARY OF THE DISCLOSURE

The disclosed system and method can provide a more efficient and reliable approach and methodology for cardiac arrhythmia analysis and treatment with better precision for characterizing arrhythmia occurrence time, position, tissue volume and energy amount by using an single catheter that integrates both EP monitoring and optical energy ablation functions. Concurrently, the disclosed multi-channel optical ablation strategy may be used with better arrhythmia mapping, signal fidelities, pathology severity & priority analysis, and ablation strategies.

A cathether system is disclosed comprising: a catheter having a distal end, a proximal end, and an interior portion. The catheter may further comprise an electrophysiology (EP) lead disposed within the interior portion, the EP lead configured for obtaining cardiac signal data. An optical lead may be disposed within the interior portion, the optical lead configured for transmitting laser radiation. The catheter may also comprise an EP signal sensor connected to said EP lead, and an optical ablator connected to said optical lead, wherein the EP signal sensor and the optical ablator are located directly adjacent to each other.

A method for sensing and ablating patient cardiac tissue is also disclosed. The method may comprise: providing a catheter having a distal end, a proximal end, an EP signal sensor located at a point between said distal and proximal ends and an optical ablator located adjacent said EP signal sensor; positioning the distal end of the catheter adjacent to patient cardiac tissue; obtaining a signal from said patient cardiac tissue using said EP signal sensor; using said obtained signal to identify a targeted portion of the patient cardiac tissue to ablate; and ablating said targeted portion of the patient cardiac tissue using said optical ablator.

### BRIEF DESCRIPTION OF THE DRAWINGS

The accompanying drawings illustrate preferred embodiments of the disclosure so far devices for the practical application of the principles thereof, and in which:

FIG. 1 shows a novel catheter system integrating ablation and EP functions for cardiac arrhythmia treatment;

FIG. 2 shows an exemplary embodiment of the integrated catheter of FIG. 1 equipped with EP signal sensor, optical ablator and lead temperature monitoring thermister;

FIGS. 3a and 3b show an ablation application comparing cardiac arrhythmia and tissue treatment before (FIG. 3a) and after (FIG. 3b) optical energy ablation;

FIG. 4a shows atrial fibrillation structure and arrhythmia distribution in the heart, while FIG. 4b shows an EP mapping and pattern analysis-based optical ablation; and

FIG. 5 shows a novel multi-branch (i.e., basket) EP/ablation chamber catheter.

### DETAILED DESCRIPTION

Monitoring of cardiac tissue and electrophysiological activities using an EP catheter are important steps in the diagnosis and pathology characterization of a patient's heart. For cardiac tissue treatment, however, additional medical devices are needed. One such additional medical device is an ablation catheter, which can be used to destroy pathological conduction tissue of the heart to return the heart to a healthy rhythm and/or to prevent an unhealthy heart rhythm. The present disclosure provides a new device and technique which integrates into a single catheter the functions of an EP catheter and an optical ablation catheter. This combined catheter may greatly improve the reliability and accuracy of the medical diagnosis and treatment of heart arrhythmias and malfunctioning tissue.

1. EP Catheter and Optical Ablation Integration

The disclosed EP catheter system integrates EP signal sensing/recording and optical (laser) ablation in a single cathether. The EP signal sensing, analysis and optical ablation system may be constructed as a closed-loop (i.e., integrating signal sensing, analysis and treatment) for automatic monitoring and arrhythmia treatment This closed-loop strategy can be further developed into automated medical treatment, in which signal and analysis feedback modules provide accurate information to treatment devices, and the treatment can be delivered to the patient in real time.

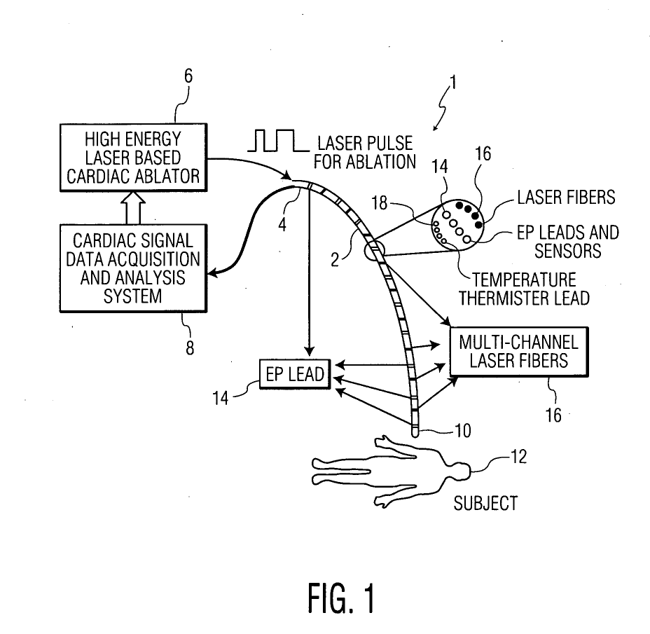

Referring to FIG. 1, the new ablation and EP catheter system 1 for cardiac arrhythmia treatment is shown. The catheter 2 may have a proximal end 4 connected to an ablation control system 6 and a data acquisition and analysis system 8, and a distal end 10 configured to be insertable into a patient 12. The catheter 2 may incorporate a plurality of leads 14, 16, 18, each designed to support a particular functionality. Thus, the catheter 2 may include one or more EP leads 14 for EP signal recording and monitoring, and one or more multi-channel laser fibers 16 for heat and laser-based ablation. The illustrated embodiment shows four of each type of lead, though a greater or fewer number could also be provided. In the illustrated embodiment, the integrated EP/ablation catheter 2 also comprises a plurality of temperature monitoring thermister leads 18 which facilitate real time continuous temperature and heat detection and analysis to enhance the EP signal recording/diagnosis and optical ablation treatment.

Typically, intra-cardiac activities are monitored using an EP catheter—for example in atrial fibrillation cases—intra-cardiac electrograms are acquired and diagnosed by a cardiac signal data acquisition and analysis system 8. The results (both the pathology of the cardiac tissue and mapping of the arrhythmia localization) are then transferred to the user or to an ablation mapping and control system (not shown). Based on the received information, the user or the ablation central processor can then decide which ablation parameters, such as pathological priorities, energy amount, time, etc., should be applied to the patient tissue. For example, the processor (executing suitable instructions) could prioritize ablation targets based on which target has the highest sensed “energy” from the associated EP sensor. Typically, the highest energy abnormality or rotor will be ablated first, followed by the next highest, etc. In addition, suitable ablation energies can be pre-programmed based on the “energy” of the associated tissue target. These criteria could be called up from a table, calculated from a suitable formula, or manually determined by the user. Estimates of the frequency or energy of the signals can be obtained using a Fast-Fourier Transform (FFT) technique. In addition, any of a variety of well known analyses can be used for the signal diagnosis, characterization and signal mapping in order to obtain the severity of the individual tissue abnormalities so that ablation priorities can be set. Examples of such analyses include signal entropy theory, symbolic complexity theory, and the like.

In one exemplary embodiment of the present disclosure the catheter 2 is a combination of a multi-channel EP lead system and multi-point ablation lead system. Compared with typical clinical procedures for cardiac arrhythmia treatment, such as AF ablation, which requires two catheters (an EP signal acquisition catheter and an ablation catheter), the new catheter is a single unit that incorporates both functions: monitoring and treatment. Additionally, compared to current electrical ablation procedures, the optical ablation procedure in this exemplary embodiment can be safer than RF ablation techniques because there is no ablation voltage and current leakage associated with the optical system. Typically, RF ablation delivers high power and energy (i.e., an electrical shock) to the cardiac tissue to eliminate targeted abnormal arrhythmia and pathology rotors. Unavoidably, this electrical shock may “leak” to the EP signal recording catheter, since cardiac tissue and blood are good conducting materials. This has two effects: (1) it results in noisy and distorted EP signals, and (2) the leakage current from the electrical shock can harm adjacent cardiac tissue in areas that are not intended to be ablated. The disclosed system does not suffer from these problems because it utilizes optical energy rather than an electrical shock, thus greatly decreasing noise and risk to the patient.

In addition, the disclosed system is more efficient than current systems due to its more precise pathological tissue localization and ablation point(s), and is therefore more reliable. The user will precisely locate the abnormal tissue via the EP signal recording/monitoring function. The ablator will then operate at the exact same location to eliminate the arrhythmia. This will do three things: (1) provide precise location for ablation because the EP sensor and the ablator are located directly adjacent to each other on the catheter, (2) is more time efficient than current systems that use multiple positioning steps, and (3) provide enhanced ablation effectiveness due to the increased likelihood that the targeted tissue will be effectively ablated. These advantages are realized because the time and energy required for ablation power delivery are related to the severity of the pathology on the corresponding accurately identified tissue points, and because there is no RF or other electrical noise or artifacts associated with optical (laser) ablation.

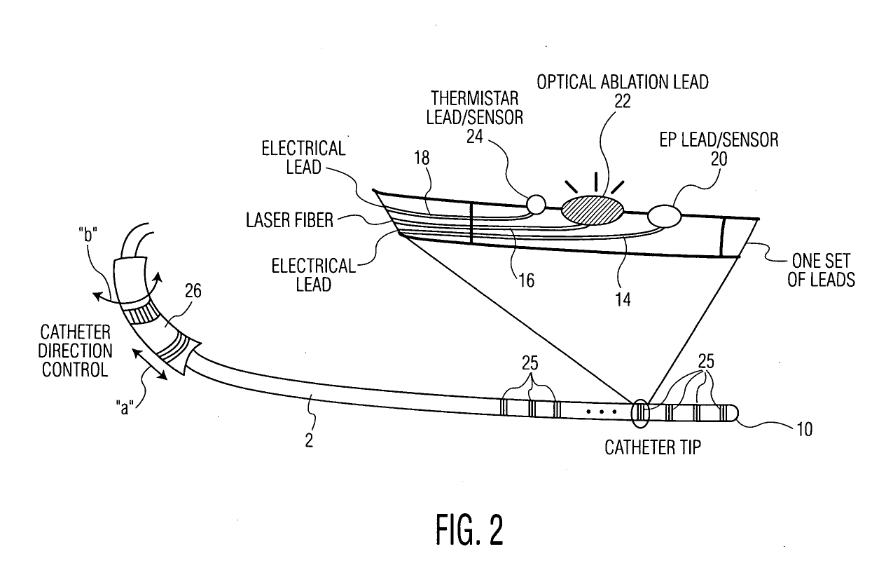

When combined with an appropriate thermister 24 (FIG. 2), the disclosed EP/ablation catheter can continuously monitor the heart energy and temperature when optical ablation energy is delivered to abnormal patient heart tissue or arrhythmia rotor(s). FIG. 2 shows the single set of lead designs of the new EP/ablation catheter 2. In the FIG. 2 embodiment, an exemplary design of the single integrated catheter 2 is equipped with EP signal sensors 20, optical ablators 22 and lead temperature monitoring thermisters 24. As can be seen, in the illustrated catheter 2, the signal sensor/lead 14, temperature sensor lead 18 and ablation lead 16 are all physically contained within the catheter 2, which can better guarantee that the desired ablating point 22 is directed precisely at the malfunctioning tissue (i.e., the tissue having the abnormal EP signals and activities). In addition, the temperature thermister 24 can continuously monitor the heat and energy delivered to the heart tissue to protect surrounding tissue during the ablation procedure. The catheter tip 10 can be controlled and steered axially “a” and rotationally “b” using a catheter direction control 26, located on or adjacent to the catheter handle.

FIG. 2 shows seven “groups” 25 of sensors 20, 22, 24 positioned at discrete intervals along the length of the catheter 2. In practice, it is expected that a catheter will be provided with three (3) to ten (10) of such groups, although any number can be provided. Spacing between adjacent groups may be from about 5 millimeters (mm) to about 10 mm, though other spacings are also contemplated.

The disclosed catheter 2 may enable a significant reduction in the total time required to perform an associated medical procedure, and may also improve ablation efficiency (i.e., higher ablation success rate and more accurate ablation localization) with more precise therapy and continuous safety monitoring of intracardiac electrograms. The disclosed catheter may also facilitate simultaneous multi-point ablation and programmable sequential multi-point ablation, which is a new method and strategy for persistent AF ablation that may greatly decrease the possibility of the secondary cardiac tissue injury. In one example, when monitoring multiple EP signals with the disclosed catheter, several channels (e.g., channels 1, 3 and 8) may evidence a similar arrhythmia mode occurring in the monitored tissue at the same time. In such a case, simultaneous ablation at all three locations may be an efficient solution. Alternatively, if those channels (1, 3 and 8) show that the arrhythmia occurs in the tissue locations sequentially (from 1 to 3, then to 8), the ablation may best be performed sequentially, saving time and ablation energy (reducing overall burning risk) with an increased treatment success rate.

The catheter 2 described in relation to FIG. 2 may further include a temperature control system to protect the myocardium during ablation procedure. Such a temperature control system may comprise a liquid perfusion media for heating and/or cooling the myocardial tissue to help keep the temperature relatively constant during the therapeutic procedure. Temperature control is important because it can control overheating of tissue during the ablation procedure, thus decreasing risk to patient tissue and the patient's life. Usually, a temperature measuring thermister is used to measure temperature of the local tissue (i.e., near the ablation point), and transfers representative data to the to a temperature comparison and control module disposed at the proximal end of the catheter. In one embodiment, the temperature comparison and control function is accomplished using appropriate software. Alternatively it can be manually controlled by the user. Overheating or burning will trigger a cutoff in the ablation circuit or treatment temporarily.

2. Cardiac Ablation Based on EP Signals

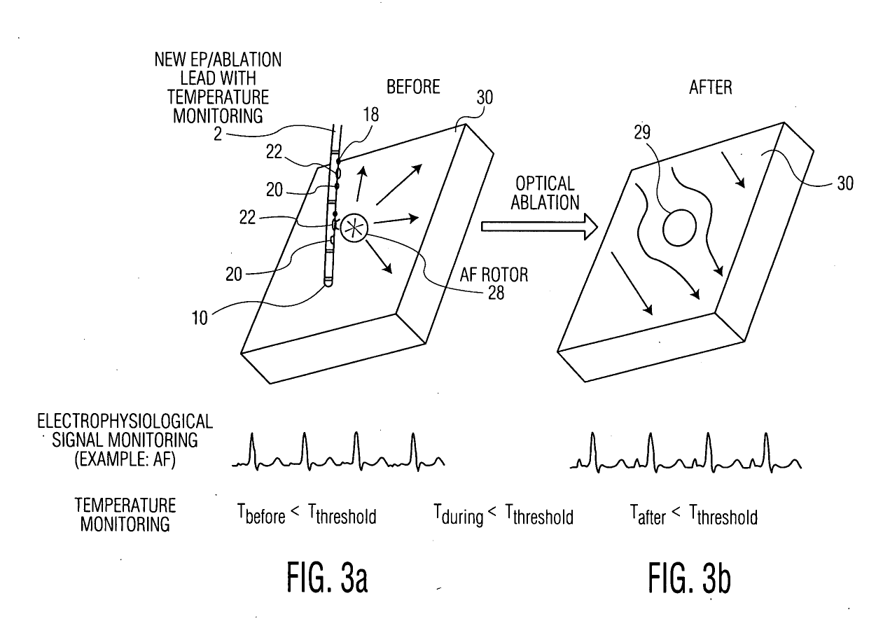

The new EP/ablation/temperature catheter may further include signal sensing, tissue/pathology mapping, temperature monitoring, and ablation treatment capabilities. FIGS. 3a and 3b illustrate an ablation application and comparison of the cardiac arrhythmia and tissue treatment before (FIG. 3a) and after (FIG. 3b) optical energy ablation using the disclosed catheter 2.

The EP/ablation catheter 2 can sense and detect abnormal electrophysiological activities. For example, during an EP procedure, one or more EP sensors 20 may obtain signals that identify an area of heart tissue that cannot conduct the pacing excitation in the correct direction but instead distributes the excitation in every direction (known as a “fibrillation rotor”). Using the EP signal monitoring and localization capabilities of the disclosed catheter 2, the optical ablating energy may be accurately delivered to the corresponding malfunctioning tissue by the associated ablator 22 without time delay. At the same time, the temperature of the corresponding cardiac tissue may be continuously monitored using the thermisters 18 to ensure patient and tissue safety such that tissue temperature (T_{before}, T_{during}, T_{after}) always remains below a predetermined threshold value (T_{threshold}). As shown in FIG. 3b, after ablation the affected tissue 29 no longer exhibits the fibrillation rotor, and only the pacing excitation in the desired direction remains.

The exemplary embodiment of the present disclosure includes both laser optical energy-based heating and cooling techniques. The technique underlying laser-base heating is well known. Laser-based cooling is accomplished by adjusting laser power and position to slow movement of the cardiac tissue at the atomic level, thus lowering the tissue temperature. The technique consists of preparing an ensemble of paramagnetic atoms in an external magnetic field in the lowest Zeeman sublevel using optical pumping, then waiting for collision-induced thermal repopulation of the higher sublevels (which proceeds at the expense of kinetic energy of the atoms), and finally optically pumping the atoms back into the lowest Zeeman sublevel, thus cooling the spin system (the emitted photons show higher energy than the absorbed photons). High temperature burning and low temperature freezing-based energy delivery and absorption can be utilized according to the type of cardiac arrhythmias and medical convenience.

3. EP Catheter Mapping Technology and Automatic Optical Ablation Strategy.

a. Single EP/Ablation Catheter

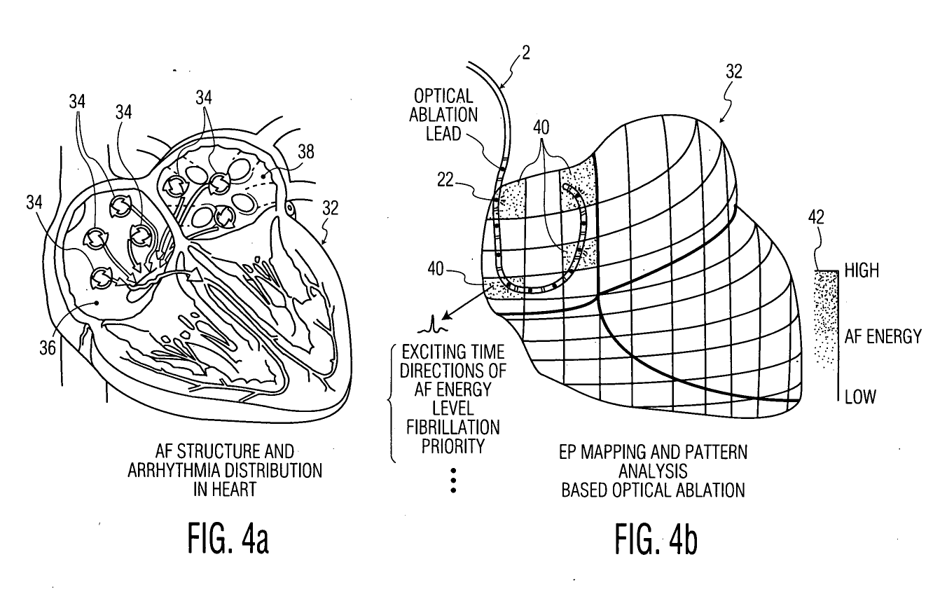

Cardiac arrhythmias and pathological mechanisms are complex and often may not be easily terminated. For example, during an atrial fibrillation case, there are usually multiple fibrillating rotors within one atrial chamber (see FIG. 4a), which can leads to a complicated situation when considering the appropriate ablation strategy to employ. Thus, questions arise such as: which rotor should be ablated first, what is the optimal sequence for the multi-rotor case requiring multi ablation, and how much energy should be applied to each ablation point (since traditional RF ablations are usually hard to analyze and thus can result in over-energy ablation). While relatively simple RF ablations may be utilized effectively for single point ablations, multi-rotor cardiac arrhythmias can require vast amounts of time and are procedurally complex, which invariably leads to increased patient risk. The disclosed system may enhance the user's ability to make decisions in the face of such complexities, with an attendant reduction in patient risk.

FIG. 4a shows an exemplary AF structure and arrhythmia distribution in heart tissue 32. The exemplary heart tissue 32 in this figure has multiple atrial fibrillation rotors 34 in both right and left atrial chambers 36, 38. With the EP sensor 20 in the disclosed catheter 2, the EP activities and arrhythmia priorities can be accurately characterized and a programmable optical ablation sequence can be achieved for real time energy deliverance. Of course, programming of the ablation sequence will occur based upon the particulars of the individual ablation case. Relevant parameters include EP signal analysis, diagnosis and characterization, the delivery local position sequence (i.e., which tissue locations will be ablated in what sequence), ablation energy for each spot, duration of energy application for each ablation spot, etc. These parameters can all be programmed and controlled individually. This programmability provides advantages of reduced procedure time and enhanced precision, as compared to current devices in which the catheter must be moved from ablation point to ablation point, thereby increasing the success rate of the ablation procedure. This will decrease the time and complexity of this therapeutic treatment since fewer catheters and catheter movements are required.

FIG. 4b illustrates the EP signal mapping and pattern analysis that can be achieved using the EP/ablation catheter system. In this figure, based upon the signal diagnosis from the EP sensor(s) 20, four (4) atrial fibrillation rotors 34 are detected in the right atrial chamber 36. These four rotors constitute the four ablation points that will be programmed for optical ablation. Signals obtained from the EP sensor(s) provide EP information and characterization of the heart tissue, such as excitation time, direction of AF rotor, energy level estimation for optical ablation and fibrillation priorities. This information can be derived by an automated algorithm or by user input.

The information obtained from the EP sensors can be converted and analyzed using well known techniques. The information obtained from the EP sensors can be used, as previously described, to prioritize the sequence of targets to be ablated, as well as the energy level (i.e., severity) of the ablation at each abnormal tissue point. The shaded portion in the EP signal and catheter mapping of FIG. 4b depicts the AF inharmonic and reentrant points, with shaded portions 40 (corresponding to the AF rotors 34 of FIG. 4a) indicating the energy level corresponding to the key 42 at the right. As previously noted, ablation priority may depend upon the severity of the arrhythmia at different locations within the heart tissue. For example, arrhythmia rotors evidencing very high energy levels (as measured by the EP sensors) may be given a high priority for ablation in the sequence due to their severity. Once these high-energy rotors are ablated, then the lower-energy rotors can be ablated, again in order of their relative severity.

As will be appreciated, tissue mapping and/or real time position localization of the catheter may be very helpful to the user. With the device of the present disclosure, since every EP signal can be acquired and derived in real time, 2D or 3D mapping and pattern analysis can be displayed in real time in the manner shown in FIG. 4b. The EP signal may be sensed, transferred to the analysis module, converted and analyzed in real time. For an exemplary 10-channel catheter, the analysis module can derive and quantify the signal in real time for characteristics such as severity, ablation time, ablation energy, and the like. The catheter position and real time cardiac function signals are transferred to a computer and display for the user to monitor. Examples of a appropriate imaging devices for accomplishing this real time 2D/3D mapping and pattern analysis are the Siemens ARTIS image system and Biosense Carto image system. Such systems may be employed to map the catheter and EP signals corresponding to tissue location. By diagnosing the position, severity, etc., the optical ablation strategy can be plotted and accomplished in real time (or it can be accomplished manually by the user).

b. Basket (Multi-Branch) EP/Ablation Catheter

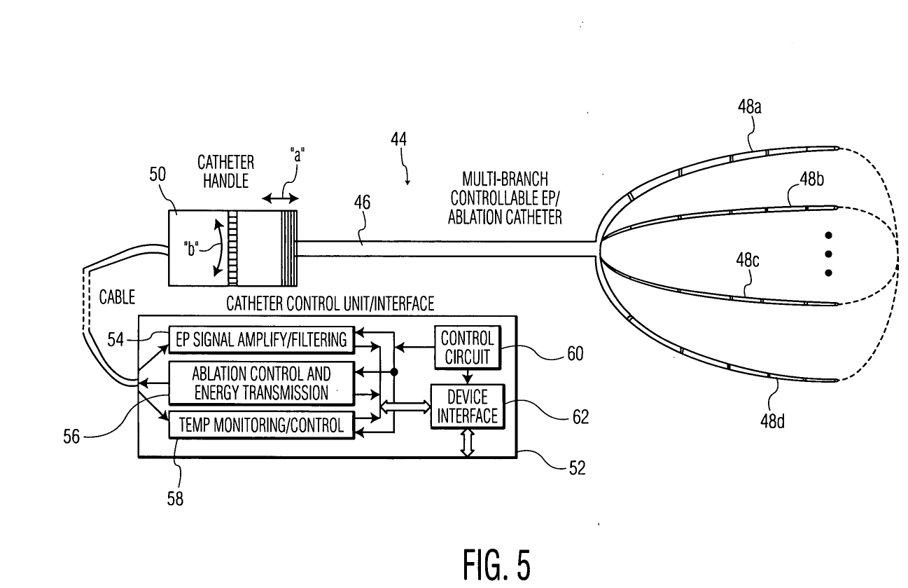

As previously noted, the disclosed catheter 2 can accomplish multi-point treatment simultaneously or sequentially. In order to more effectively achieve multi-point arrhythmia treatment, especially for applications in which 3D tissue mapping and pattern analysis-based ablations are performed, a multi-branch (basket) EP/ablation catheter system 44 shown in FIG. 5 may provide enhanced convenience and versatile functions during medical treatment, such as during chamber-based multi-point simultaneous ablation.

The novel basket (multi-branch) EP/ablation catheter system 44 for 3D-based mapping, pattern analysis and ablation, may comprise a basket catheter 46 having multiple branches 48a, 48b, 48c, 48d on which are disposed multiple leads for EP signal monitoring, ablation delivery, and temperature monitoring, as previously discussed in relation to FIGS. 1-4b. Each branch 48a-d of the basket EP/ablation catheter can be independently steered and controlled both axially “a” and rotationally “b” using a catheter handle 50. A control unit/interface 52 may be provided between the basket catheter 46 and a variety of hardware devices, as an ablation generator and/or and EP monitor. The control unit/interface 52 may comprise appropriate circuitry for EP signal amplification and filtering 54, ablation control and energy transmission 56, as well as temperature monitoring and control 58. The control unit/interface 52 may further comprise a master control circuit 60 and a device interface 62, which can be utilized for functions such as exchanging data between the catheter control interface 52 and hardware devices. Examples of such hardware devices include wired or wireless technologies (Ethernet, Blue Tooth) for data communications, command and control, and data compression (since very large quantities of data may be obtained during the data acquisition phase). Additionally, a real time display or embedded system can be integrated into the catheter system 1, for example, a mini-LCD for multi-channel display for EP signals and ablation information (e.g., temperature, energy, duration, etc.)

In use, the control unit/interface may be operated to control the EP/ablation catheter, provide EP signal conditioning and transmission, perform temperature monitoring, perform ablation control (e.g., by programming the required energy and time for a particular ablation) as well as providing safety control (e.g., patient safety protection, such as ablation cutoff control).

In addition to the features and capabilities expressly identified above, the various embodiments of the present disclosure may also include:

(a) A catheter steering and control unit, such as a handle for catheter positioning and tuning.

(b) An interface unit for electrical and optical conditioning, filtering and transmission.

(c) Programmable simultaneous and sequential 3D-based ablation strategy (e.g., prioritization of ablation points, delivery energy, ablation duration, etc.) based on 2D or 3D mapping and signal analysis and diagnosis; and

(d) Continuous temperature monitoring-based safety strategy for ablation application. This strategy is implemented to enhance patient safety in order to avoiding overheating and burning of tissue. Continuous and real time temperature monitoring and control can greatly reduce patient and tissue risk by cutting off the circuit, decreasing ablation energy, and the like, if an over-temperature condition is sensed.

As disclosed, the system and method of the present disclosure can be used to monitor, diagnose and treat all arrhythmias types (atrial fibrillation, atrial flutter, AV re-entrant tachycardia, accessory pathway arrhythmias (such as Wolff-Parkinson-White syndrome (WPW)) and ventricular tachycardia) that are currently treated using traditional EP treatment catheter sets, but with greater precision & patient safety and less procedure time and case complexity as compared to current devices.

The disclosed system and method may solve the shortcomings and improve the performance of current cardiac arrhythmia analysis and ablation techniques and strategies. In summary, the disclosed system may provide the following advantages over the current cardiac monitoring strategies:

(1) It may greatly reduce medical procedure time, case complexity and patient risk compared with the traditional method of inserting two catheters (one for EP signal acquisition and one for ablation) into the patient's heart and circulation system.

(2) It may lead to more precise positioning and greater timing precision as compared to current systems.

(3) Compared with traditional ablation strategies, the disclosed system may provide the following advantages:
(i) Utilizing optical ablation results in no additional electrical noise or artifacts that could affect EP signal monitoring.
(ii) Optical ablation may have a much better energy shock and delivery focus to the exact malfunctioning cardiac tissue thus leading to an increased ablation success rate.
(iii) Optical ablation energy in one embodiment has one directional focus and there is no energy loss as occurs with RF ablation.

(4) EP signal mapping and function analysis/characterization can provide real time mapping, arrhythmia pattern tracking, tissue localization, pathological size pathological priorities of the arrhythmia tissue, and energy estimation for optical ablation.

(5) The multi-channel optical/laser ablating ability for cardiac arrhythmia treatment can provide the potential for real time ablation for emerging arrhythmia and existing pathology in the cardiac tissue. Additionally multi-channel optical ablation can reduce the rate of false ablation and decrease risk of ablation application since multi-channel optical ablation sites can be more precisely and correctly linked with EP signal recording position as compared with current systems. Further, multi-channel optical ablation can achieve simultaneous multi-point ablation and programmable sequential multi-point ablation, which may be a benefit for persistent AF ablation.

(6) Adaptive and close-loop control for optical energy delivery for arrhythmia treatment based on real time electrophysiological signals feedback (signal analysis and pattern characterization) and mapping—automatic ablation may be a possibility for real time close-loop optical ablation. Flexible and multi-degree ablation ability (by lead angle and rotation control) for AF treatment both for precise tissue position, and real time termination of the pathological rotor corresponding to the EP recording signal and points

(7) The concepts of the present disclosure can be used to monitor, diagnosis and treat both atrial and ventricular arrhythmias. Furthermore, optical ablation idea in this preferred embodiment of current disclosure includes both high temperature (deliver energy to heart tissue) and low temperature (absorb energy from heart tissue) ablation methods and strategies.

The features of the system and technique have been disclosed, and further variations will be apparent to persons skilled in the art. All such variations are considered to be within the scope of the appended claims. Reference should be made to the appended claims, rather than the foregoing specification, as indicating the true scope of the subject system and technique.

What is claimed is:

1 A cathether system comprising:
a catheter having a distal end, a proximal end, and an interior portion;
an electrophysiology (EP) lead disposed within the interior portion, the EP lead configured for obtaining cardiac signal data;
an optical lead disposed within the interior portion, the optical lead configured for transmitting laser radiation; and
an EP signal sensor connected to said EP lead, and an optical ablator connected to said optical lead;
wherein the EP signal sensor and the optical ablator are located directly adjacent to each other.

2. The catheter system of claim 1, further comprising a thermister lead disposed within the interior portion and a thermistor connected to the thermister lead, the thermistor positioned adjacent the EP signal sensor and the optical ablator to sense a temperature of patient tissue during an ablation procedure.

3. The catheter system of claim 1, wherein a plurality of EP leads and a plurality of optical leads are disposed within the interior portion, and a plurality of EP signal sensors and a plurality of optical ablators are connected to respective ones of said plurality of EP leads and said plurality of optical leads, and wherein pairs of EP signal sensors and optical ablators are positioned at discrete intervals along a length of said cathether to enable EP signal sensing and optical ablating at a plurality of locations.

4. The catheter system of claim 3, further comprising a cardiac signal data acquisition and analysis system connected to said catheter for receiving and analyzing signals from at least one of said plurality of EP signal sensors.

5. The catheter system of claim 1, wherein the cathether comprises a plurality of branches, each branch containing at least one said EP lead, at least one said optical lead, at least one said EP signal sensor connected to said at least one EP lead, and at least one said optical ablator connected to said at least one optical lead; wherein each of the plurality of branches is position controllable using a control handle.

6. The catheter system of claim 5, wherein the EP signal sensors of said plurality of branches are operable to acquire cardiac signal data and the optical ablators of said plurality of branches are operable to perform ablation of multiple tissue locations either serially or simultaneously.

7. The catheter system of claim 6, wherein each of said plurality of branches contains a thermister lead and a thermistor connected to the thermister lead, the thermistor positioned adjacent the EP signal sensor and the optical ablator of that branch to sense a temperature of patient tissue during an ablation procedure.

8. The catheter system of claim 1, further comprising a control unit configured to: (a) amplify and condition EP signals received from the EP signal sensor, and (b) control an amount of energy supplied to the optical ablator.

9. The catheter system of claim 8, further comprising a display for presenting a graphical representation of said EP signals to a user.

10. The catheter system of claim 1, wherein the optical ablator is configured to heat tissue.

11. The catheter system of claim 1, wherein the optical ablator is configured to cool tissue.

12. A method for sensing and ablating patient cardiac tissue comprising:
providing a catheter having a distal end, a proximal end, an EP signal sensor located at a point between said distal and proximal ends and an optical ablator located adjacent said EP signal sensor;
positioning the distal end of the catheter adjacent to patient cardiac tissue;
obtaining a signal from said patient cardiac tissue using said EP signal sensor;
using said obtained signal to identify a targeted portion of the patient cardiac tissue to ablate; and
ablating said targeted portion of the patient cardiac tissue using said optical ablator.

13. The method of claim 12, further comprising providing a thermistor adjacent the EP signal sensor and the optical ablator to sense a temperature of patient tissue during an ablation procedure.

14. The method of claim 12, wherein said catheter comprises a plurality of EP signal sensors and a plurality of optical ablators, and wherein pairs of said EP signal sensors and said optical ablators are positioned at discrete intervals along a length of said cathether to enable EP signal sensing and optical ablating simultaneously or in series at a plurality of patient tissue locations.

15. The method of claim 12, wherein the cathether comprises a plurality of branches, each branch containing at least one said EP lead, at least one said optical lead, at least one said EP signal sensor connected to said at least one EP lead, and at least one said optical ablator connected to said at least one optical lead; wherein each of the plurality of branches is position controllable using a control handle.

16. The method of claim 15, further comprising operating the EP signal sensors of said plurality of branches to identify a plurality of targeted tissue locations and operating at least two of the optical ablators of said plurality of branches to ablate of multiple tissue locations either simultaneously or in series.

17. The method of claim 16, wherein each of the plurality of branches comprises a thermistor positioned adjacent an associated EP signal sensor and optical ablator, the method further comprising sensing a temperature of patient tissue using at least one of the thermistors during an ablation procedure.

18. The method of claim 12, further comprising using a control unit to: (a) amplify and condition EP signals received from the EP signal sensor, and (b) control an amount of energy supplied to the optical ablator.

19. The method of claim 18, further comprising presenting a graphical representation of said EP signals to a user via a display.

20. The method of claim 19, wherein the ablating step comprises heating said targeted portion of the patient cardiac tissue.
